(19) United States
(12) Patent Application Publication
(10) Pub. No.: US 2025/0099003 A1
(43) Pub. Date: Mar. 27, 2025

(54) RETAINING DEVICE FOR RETAINING A WEARABLE MONITORING DEVICE

(71) Applicant: UNEEG MEDICAL A/S, Lynge (DK)

(72) Inventor: Klaus IPSEN, Vanloese (DK)

(73) Assignee: UNEEG MEDICAL A/S, Lynge (DK)

(21) Appl. No.: 18/291,439

(22) PCT Filed: Jul. 14, 2022
(86) PCT No.: PCT/EP2022/069708

§ 371 Date: Jan. 23, 2024

(51) Int. Cl. A61B 5/24; A61B 5/00

(52) U.S. Cl. CPC A61B 5/24; A61B 5/6833

(57) ABSTRACT

A retaining device for retaining a wearable monitoring device for gathering a bio-electrical signal from a body region of a person. The retaining device has a holding frame having a baseplate and retaining mechanism for reversibly retaining the monitoring device onto the baseplate, and preferably an adhesive layer attached at least to a part of the baseplate for fixation of the holding frame onto the skin of the person. An area covered by the baseplate is reduced with respect to an area covered by the monitoring device for ventilation of the skin during wearing the monitoring device.

The present invention relates to a retaining device for retaining a wearable monitoring device, especially to a body region of a person. The monitoring device is preferably adapted for gathering a bio-electrical signal from that person. The invention relates also to a monitoring system.

Bio-electrical signals are here understood to be electrical potential differences across a tissue, organ or cell system. The best known examples for application or utilization of such signals are Electrocardiogram (ECG), Electroencephalogram (EEG) and Electromyogram (EMG). A (personal) wearable monitoring device denotes especially a device adapted for long term monitoring, e.g. several days, months or even years, where the person can live a normal life without having to pay more attention to the monitor than necessary with a pair of glasses or a hearing aid. The monitoring may be for purposes of surveillance of a condition of the person and/or for providing some kind of alarm or information in case predetermined conditions are met. The monitor may also be applied for collection of data for further analysis, e.g. for diagnostic purposes or for research use.

An example of monitoring bio-electrical signals is the recording and analysing of an EEG signal for various diagnostic purposes. WO 2006/047874 A1 describes measurement of brain waves particularly for detecting the onset of an epileptic seizure.

EEG monitors may also be applied for surveillance of persons having diabetes, where low blood sugar levels may cause hypoglycaemic attacks. A system for surveillance of the EEG signal where changes may indicate an imminent hypoglycaemic attack is disclosed in WO 2006/066577 A1.

Especially for long term monitoring, it is known to make use of implants to pick up the bio-electrical signals. These implants feature a transmission unit to send the picked up signals to a recording unit that resembles the actual monitoring device. Such a system is known for example from WO 2015/144214 A1.

The implant in such systems is regularly provided with energy by the monitoring device via wireless energy transmission. In order to keep the energy need by the implant low, a transmission distance, that is as short as possible, is the target. Therefore, the monitoring device is usually attached directly over the implant, which in turn may preferably be placed extracranially, having only the skin between the implant and the monitoring device.

Furthermore, the monitoring device should be as small as possible in order to be as discreet as possible when worn. The energy for the own operation as well as or the implant is usually stored in a battery, in most cases a secondary battery cell or “accumulator”. In order to charge the battery the monitoring device usually has to be removed from the body, in most cases at a daily interval. In the case where the monitoring device is attached adhesively to the skin, that attachment and detachment causes irritated skin.

Therefore, there is a need for retaining a monitoring device with as little irritation of the skin as possible.

Such need is met according to the invention by a retaining device according to the features of claim 1. Furthermore, such need is met according to the invention by a wearable monitoring system according to the features of claim 19. Further expedient and per se inventive embodiments and developments are disclosed in the dependent claims and the following specification.

The retaining device according to the invention is adapted to retain a wearable (especially personal) monitoring device that serves for gathering a bio-electrical signal to a body region of a person. The retaining device comprises a holding frame having a baseplate. The holding frame also comprises retaining means for reversibly retaining the monitoring device onto the baseplate. Hereby, an area covered by the baseplate is reduced with respect to an area covered by the monitoring device. That reduction of that area is intended for ventilation of the skin during wearing the monitoring device.

The term “area covered by . . . ” is understood here and in the following to mean especially the area of the respective part (here especially the baseplate and the monitoring device) projected onto the underlying surface. During intended use that surface would be especially the skin of the user. For the sake of shortness that area is also called “projected area” in the following.

Due to the ventilation of the skin by means of the reduced projected area of the baseplate it is possible to reduce skin irritation even for long term applications. Furthermore, due to the retaining means for reversibly retaining the monitoring device to the body of the user, the monitoring device, that is preferably driven by a secondary battery cell (i. e. a rechargeable battery), may be detached from the body for charging, servicing, readout or else without detaching an adhesive layer from the skin of the user. Such adhesive layer is expediently used for fixation of the holding frame on the skin of the user. For monitoring 24 hours a day, the user may have at least two monitoring devices (or twice the number needed to be worn at the same time), so that at least one monitoring device may be recharged, while the other one monitoring device is in use (i. e. is worn).

In a preferred embodiment a ventilated area which is not covered by (especially massive parts of) the baseplate has at least a portion of 25 percent with respect to the area covered by the monitoring device (i. e. the projected area of the monitoring device). That ventilated area is for example the difference of the projected area of the monitoring device and the projected area of the baseplate. Expediently, the ventilated area is at least 25 percent of the projected area of the monitoring device and, preferably, a maximum of 85 percent. More preferred, the ventilated area is between 30 and 80 percent of the projected area. Expediently, that ratio is between 40 and 75 percent, more expediently between 50 and 70 percent and most preferred at about (i.e. +/−5 percent) 55 percent.

In an appropriate embodiment the baseplate has a (outer) shape that is at least roughly approximated to the outer contour of the monitoring device. At least roughly approximated is to be understood to mean especially that a part, preferably a major part (i. e. more than 50 percent of the area covered by the baseplate) exhibits an outer shape that corresponds to the monitoring device.

Especially in the case above, the holding frame, in a further expedient embodiment, has a number of apertures penetrating (or perforating) the baseplate for ventilation of the skin.

Expediently, the number of apertures is more than one.

For example, (described from another perspective) the relation of the projected area of the whole baseplate (i.e. especially without consideration of the apertures) to the ventilated area, in that embodiment preferably the area of the number of apertures, is (during intended use) between 6 and 1,5, in particular between 5,5 and 1,5, expediently between 3 and 1,5, preferably between 2,25 and 1,8, especially between 2,1 and 1,9. For example, the area covered by the baseplate is about 720 mm² and the area of the number apertures is about 130 mm². Thus, the area of the number of (preferably more than one) apertures is especially as large as possible while also maintaining mechanical stability of the baseplate.

In one example the baseplate is perforated by multiple (especially likewise) wholes, e.g. circular bores.

According to another embodiment the baseplate has a skeleton-like or meshed structure of interconnected bars (or “webs”) outlining the number of apertures. Specifically, the apertures, in that embodiment, are of an oval and/or polygonal shape.

In the sense of an easy and cost effective manufacturing method (especially for a serial production), the holding frame is injection moulded from a thermoplastic material, e. g. a polycarbonate.

For example, the baseplate is just resembled by a relatively narrow ring-like frame structure or ring-like beam having a curvature corresponding to the monitoring device and outlining one aperture. For further stabilization, a further development of that example might have two apertures by means of an additional narrow beam (or web) spanning from side to side of the ring-like beam (i.e. subdividing one bigger aperture into said two apertures).

In an expedient embodiment, the retaining device, furthermore, comprises an adhesive layer (especially the one mentioned above) that is attached at least to a part of the baseplate. Said adhesive layer serves for fixation of the holding frame onto the skin of the person.

According to a preferred embodiment said adhesive layer has a multiple layer structure comprising a carrier layer attached by an adhesive sublayer to the baseplate and a contact layer that serves as a skin adhesive during intended use.

Especially, the adhesive layer is resembled by some kind of skin plaster which—by itself—is glued to the baseplate (by way of said adhesive sublayer). In a preferred version, the adhesive layer is of the kind of a usual sensitive skin plaster in order to reduce skin irritation only by wearing the plaster itself.

According to an expedient embodiment also said adhesive layer has a number of through-holes corresponding to the respective aperture of the baseplate. By that, the ventilation of the skin and, thus, the reduction of skin irritation may be enhanced. The through-holes may be (especially round) holes (“bores”) that are located within the area of the respective apertures of the baseplate they are aligned with. In that case, the through-holes are smaller than the respective aperture, thus, the area adhering to the skin is enlarged in comparison to the area of the “massive” parts (i.e. without the apertures) of the baseplate. The trough-holes are optionally made by cutting or punching the adhesive layer. Optionally, the through-holes have the same form as the respective aperture.

According to a further expedient embodiment the baseplate comprises means for ventilation of at least some of the number of apertures especially for the case that the monitoring device is attached to the holding frame. In that case, the monitoring device usually covers the number of apertures at least partially. These means for ventilation are located in a preferred version at an upper side of the baseplate facing the monitoring device during intended use, thus, facing away from the adhesive layer, if applicable.

According to a preferred embodiment the means for ventilation described above comprise at least one ventilation channel embossed especially into the upper side of the baseplate. Said at least one channel connects one of the number of apertures with a rim of the baseplate. In other words, said channel enables a sideways ventilation. During intended use of the monitoring device and of the retaining device the channel allows air to flow between the baseplate and the monitoring device to and from the aperture connected by the channel. Thus, the area under the monitoring device may be ventilated since the side of the monitoring device facing the user's skin is in fluid communication with the environment via the at least one ventilation channel. “Embossing”, in that case, is considered as locally reducing the wall thickness of the baseplate, especially irrespective of the manufacturing method.

According to a further embodiment the means for ventilation comprise (alternatively or additionally to the above ventilation channel) at least one protrusion serving as a spacer between the surface of the upper side and the surface of the monitoring device being directed to the baseplate during intended use. Preferably, there are more, e. g. two or three such protrusions.

In another expedient embodiment-alternatively or additionally to the aforementioned aperture(s)—the baseplate has a beam-like structure connecting the retaining means and leaving a number of recesses open to a border (or margin) defined by the projected area of the monitoring device. In other words, the beam-like structure has some kind of stem or twig which is relatively narrow and which is running between a number of gaps (the above recesses) that are sideways open. Thus, in a most rudimentary version there is only one beam which is narrow with respect to the width of the monitoring device (e.g. at most about half of the width of the monitoring device). The parts of the projected area of the monitoring device not covered by said beam resemble said gaps—or said aforementioned ventilated area.

According to an expedient embodiment the retaining means for retaining the monitoring device to the baseplate comprise a snap-on hook. Said snap-on hook is preferably located such that a user, i.e. the person wearing the monitoring device, is able to reach it for releasing the monitoring device. Especially in the case of an injection moulded holding frame, a snap-on hook is an easy to design and easy to use retaining mechanism. Additionally, a snap-on hook offers a haptic and/or acoustic feedback of reaching the intended locking state. Preferably, in that case, the monitoring device has an indentation that is designed correspondingly to the snap-on hook such that the latter may snap into that indentation when the monitoring device is positioned in the intended wearing position.

According to another expedient embodiment (especially as an alternative to the snap-on hook) the retaining means comprise a holding magnet. Said holding magnet is adapted to couple with a counter magnet located with the monitoring device.

According to a further expedient embodiment the retaining means comprise (especially additionally to the snap-on hook or the holding magnet) an overhanging structure into the clearing of which the monitoring device is inserted during intended use. Especially, said overhanging structure serves as a counter support or down holder acting together with the snap-on hook or the holding magnet. In that case the snap-on hook or the holding magnet and the overhanging structure are allocated in the regions of opposite ends of the baseplate.

Said overhanging structure resembles in one embodiment a shoe cap like structure. In an alternative embodiment the overhanging structure is made as some kind of rail for guiding the monitoring device over a longer distance than the shoe cap like version.

Preferably, the overhanging structure is positioned in a region of the baseplate facing upwards during intended use. That enables a protection against unintended entangling with a garment, e.g. a pullover during dressing.

According to a further preferred embodiment the snap-on hook is provided at a rim confining a heel part of the baseplate, i. e. especially the part opposite of the part having the overhanging structure. Said heel part is especially tapered with respect to a remaining main part of the base plate. Such tapering enables a higher flexibility of the heel part and, thus, of the snap-on hook.

Especially, the holding frame is roughly designed as the “colour” spades of a card game or as an arrowhead. The “stem” part of the spades being the heel part and the leaves or heart like part being the main part of the baseplate. According to one embodiment the tip part of the spades has the shoe cap like structure of the overhanging structure.

Thus, the outer shape of the baseplate and, due to that, of the holding frame are approximated to a triangle. That is, especially, since the monitoring device is itself also triangular.

According to a further expedient embodiment the retaining device additionally comprises means for preventing an unintended allocation of the monitoring device to the holding frame. In other words, there is a fool proof attaching mechanism with the retaining device.

Said means for preventing an unintended allocation of the monitoring device preferably comprise the baseplate having a curvature. Especially, such curvature is a convex bulge of the baseplate towards the monitoring device. Preferably, such curvature is adapted to a curvature of the monitoring device. In that case, a wrong allocation of the monitoring device would prevent the snap-on hook and/or the overhanging structure from getting a hold on the monitoring device.

According to an alternative (or even additional) embodiment the means for preventing an unintended allocation of the monitoring device comprise the snap-on hook having a length shorter than the thickness of the monitoring device. Additionally, the length of the snap-on hook is different to half of the thickness of the monitoring device. In other words, for preventing the unintended allocation, the snap-on hook is expediently shorter than half of the thickness of the monitoring device or longer than half of the thickness but shorter than the whole thickness. Preferably, the above mentioned indentation of the monitoring device is positioned correspondingly on the outside of the monitoring device. Further preferably, the snap-on hook is expediently shorter than half of the thickness of the monitoring device so that the snap-on hook and the indentation of the monitoring device will not be able to engage with each other if they are not placed correctly. Due to the above described length of the snap-on hook a flipped allocation of the monitoring device will not be possible because the length of the snap-on hook does not match to the indentation of the monitoring device in the latter case.

The wearable monitoring system according to the invention comprises the above described retaining device as well as the above described wearable monitoring device. Therefore, the monitoring system shows the same features and advantages as described above.

Hereinafter, embodiments of the invention are explained on the basis of a drawing. Therein is shown in

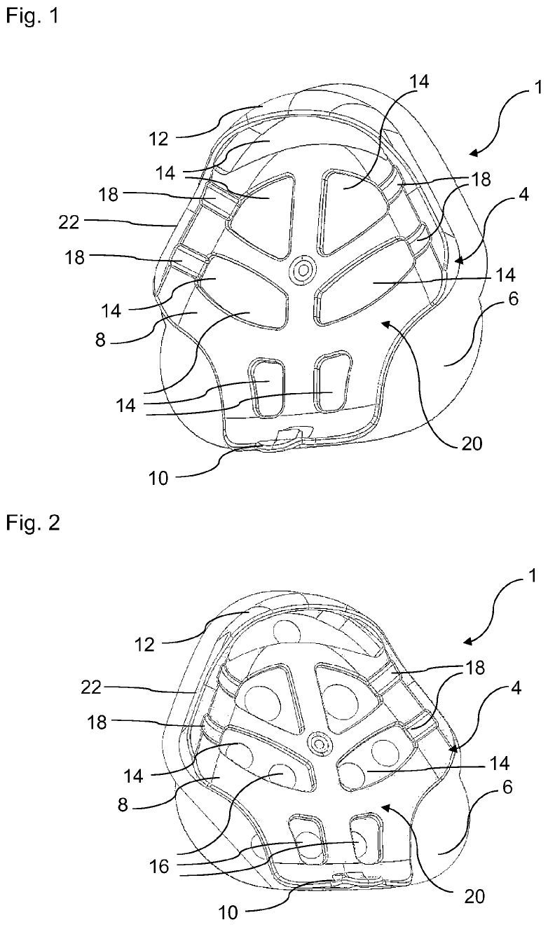

FIG. 1 in a perspective view schematically a retaining device for retaining a wearable monitoring device,

FIG. 2 in another perspective view schematically the retaining device in an intended use state,

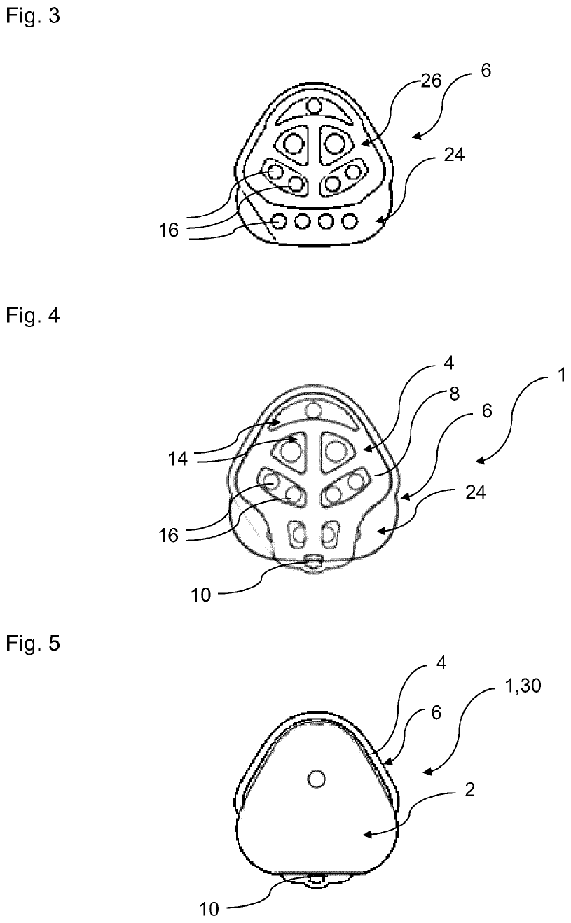

FIG. 3 in a view upon an upper side an adhesive layer of the retaining device,

FIG. 4 in a view according to FIG. 3 the retaining device in a state as in FIG. 2,

FIG. 5 in a view according to FIG. 3 the retaining device and the monitoring device attached to the retaining device,

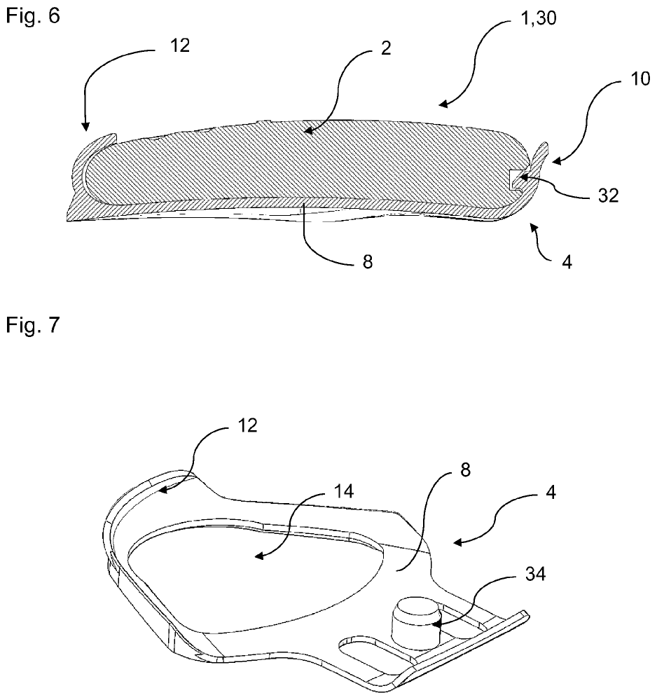

FIG. 6 in a sectional view the retaining device and the monitoring device,

FIG. 7 in another perspective view schematically a further embodiment of the retaining device, and

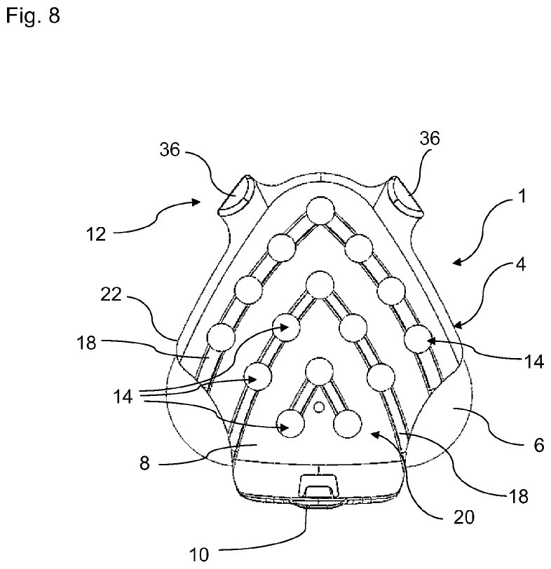

FIG. 8-12 in a view according to FIG. 3, respectively, yet further embodiments of the retaining device.

Same features are named by same reference numerals throughout all figures.

In FIG. 1 a retaining device 1 for a wearable monitoring device 2 (s. FIGS. 5 and 6) is shown. The monitoring device 2 is adapted to gather a bio-electrical signal, especially from a subcutaneous implant (not shown). The retaining device 1 is adapted to be worn on the skin over a long term that may be several days.

The retaining device 1 comprises a holding frame 4 for holding (retaining) the monitoring device 2 and an adhesive layer 6 for attachment of the holding frame 4 to the skin of a user. The holding frame 4 comprises a baseplate 8 that has roughly the shape of an arrowhead or spades. In addition to the baseplate 8 the holding frame 4 comprises means for holding the monitoring device 2.

According to the embodiment shown in FIG. 1 the means for holding the monitoring device 2 are resembled by a snap-on hook 10 and an overhanging structure 12. The latter is located at a tip of the arrowhead shape of the baseplate 8 which is worn during intended use facing upwards. The overhanging structure 12 resembles a kind of a shoe cap. The monitoring device 2 which has the shape of a triangle is pushed under the overhanging structure 12 with its tip and is then clipped onto the baseplate 8 by the snap-on hook 10.

The holding frame 4 is manufactured integrally with the baseplate 8, the overhanging structure 12 and the snap-on hook 10 by injection molding, e. g. using a polycarbonate.

In order to further the elastic features of the holding frame 4 in the region of the snap-on hook 10 the baseplate 8 is tapered (just as the foot of the arrowhead) in comparison to the upper (or main) part of the baseplate 8.

The baseplate 8 is perforated by a number of apertures 14. In the embodiment according to FIG. 1 there are seven apertures 14. The apertures 14 serve for ventilation of the underlying skin during usage of the retaining device 1 and of the monitoring device. Therefore, the adhesive layer 6 is also perforated by through-holes 16 that are located in the regions of the apertures 14 (s. FIG. 2). In FIG. 1 the through-holes 16 are not shown because the adhesive layer 6 is not ready for usage and, therefore, covered by a peel-off film (not shown).

Since the monitoring device 2 would cover most parts of the apertures 14 further means for ventilation are formed into the holding frame 4. These means are ventilation channels 18 that lead on an upper side 20 of the baseplate 8 from a rim 22 of the baseplate 8 to a respective aperture 14. The ventilation channels 18 are engraved into the upper side 20 of the baseplate.

A ventilated area, i.e. the difference of the projected area of the monitoring device 2 and the projected area of the “massive parts” of the baseplate 8 (i. e. without the area of the respective apertures 14) is about 472 mm². The projected area of the monitoring device is about 893 mm². Thus, the ventilated area is about 53 percent of the projected area of the monitoring device 2.

In FIG. 3 the adhesive layer 6 is schematically shown. The adhesive layer 6 is made up from a usual plaster 24, having for example a textile layer (woven or nonwoven, not shown in detail) that is carrying a skin adhesive which is covered before intended use by above peel-off film. On the upper side, the adhesive layer 6 comprises an adhesive film 26 (or sublayer) by which it is attached to the baseplate 8, at least to the upper part of the baseplate 8. That is shown in FIG. 4. The tapered part (or “heel part”) of the baseplate 8 is not bonded to the adhesive layer 6 via the adhesive film 26, thus, enhancing the flexibility of the adhesive layer 6, especially for conforming to the shape of the user's head.

FIG. 5 depicts schematically the monitoring system 30 made up from the retaining device 1 and the monitoring device 2 attached to it.

FIG. 6 depicts in a sectional view schematically the holding frame 4 and the monitoring device 2 in the intended use state. As it can be seen, the tip (left side of FIG. 6) of the monitoring device 2 is held beneath the overhanging structure 12. The “heel” of the monitoring device 2 is held by the snap-on hook 10 engaging in an indentation 32 of the monitoring device 2. The indentation 32 is within a lower half of the thickness of the monitoring device. The snap-on hook 10 is correspondingly short.

Furthermore, the monitoring device 2 as well as the holding frame 4 are curved. Due to that, a foolproof alignment of the monitoring device 2 is made possible. Additionally, the holding frame 4 may fit closely to the skin since the skin especially on the head of a user is not plane by nature.

FIG. 7 schematically shows a further embodiment of the holding frame 4. Here, a holding magnet 34 is used instead of the snap-on hook 10. Furthermore, there is one relatively big aperture 14 and two smaller apertures 14 beside the holding magnet 34. The ventilated area in that embodiment is about 652 mm², and, thus, the ventilated area is 73 percent of the projected area of the monitoring device 2 (the projected area of the monitoring devices 2 of the respective embodiments stays the same).

FIG. 8 schematically shows yet a further embodiment of the holding frame 4. In general, this embodiment is similar to the embodiment of FIGS. 1 and 2. However, here, the overhanging structure 12 is made of two equal parts on both sides of the tip. Thus, the overhanging structure 12 looks some kind of a pair of floppy ears 36 wherein the monitoring device 30 may be pushed beneath the tips of both floppy ears 36.

Furthermore, the apertures 14 are made circular and are aligned on a row along a respective ventilation channel 18 that connects several apertures 14 with the rim 22 of the holding frame 4.

The ventilated area in that embodiment is about 510 mm², and, thus, the ventilated area is about 57 percent of the projected area of the monitoring device 2.

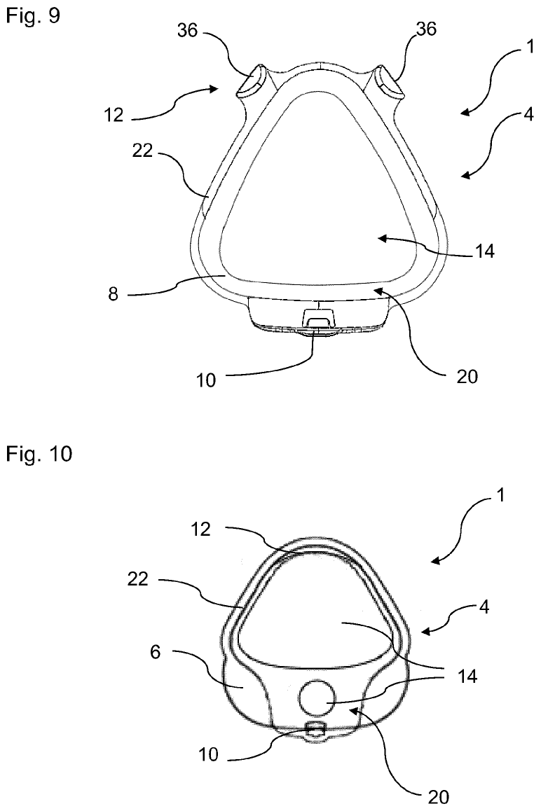

FIG. 9 displays another embodiment of the holding frame 4. The overhanging structure 12 is similar to the embodiment of FIG. 8. The baseplate 8 approximates the shape of the monitoring device 2. However, there is only one (large) aperture 14 that reduces the baseplate 8 to be only a narrow frame beam of a roughly triangular shape that surrounds the aperture 14 ring-like. In that embodiment, the ventilated area is about 72 percent of the projected area of the monitoring device 2.

FIG. 10 shows another embodiment of the holding frame 4. The outer shape of the baseplate 8 corresponds to the one of FIG. 3, but having only two apertures 14, one similar to the one of FIG. 9. The other one being a circular bore in the tapered part (or “heel part”) of the baseplate 8. In that embodiment, the ventilated area is also about 72 percent of the projected area of the monitoring device 2.

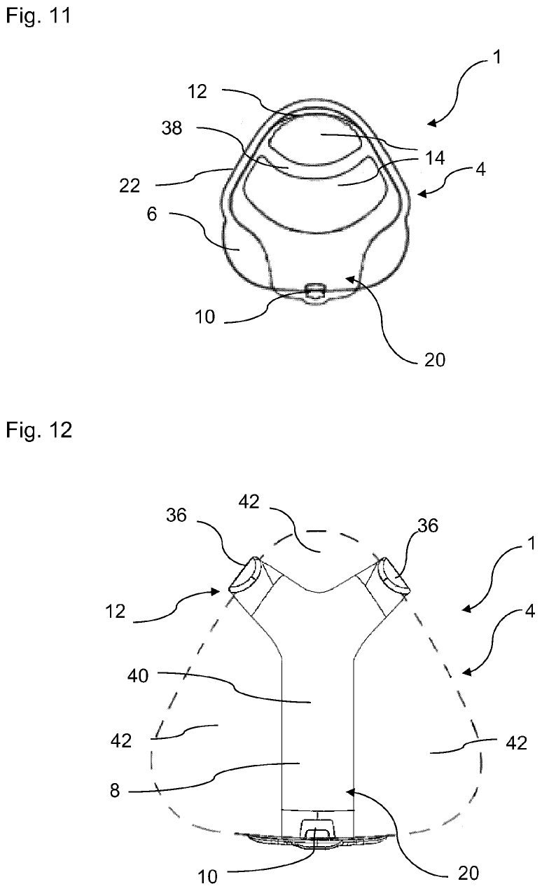

In a yet another embodiment, shown in FIG. 11, a narrow beam or web 38 runs from side to side of the narrow frame beam which is similar to the one of FIG. 9 such that two apertures 14 are shaped. In that embodiment, the ventilated area is about 66 percent of the projected area of the monitoring device 2.

FIG. 12 shows another alternative embodiment of the baseplate 8. The baseplate 8 resembles a stem 40 (or a “central web”) connecting the overhanging structure 12 and the snap on hook 10. Spoken figuratively, the baseplate 8 is of a tree-like shape having the stem 40 branching into two “twigs” connection the floppy ears 36. In that case, the ventilation is realized by the reduced area of the baseplate 8 with respect to the area of the monitoring device 2 (indicated by a dashed line). The stem 40 (and its twigs connecting the floppy ears 36) run in other words between gaps 42 (or recesses) that are open to the outside with respect to the outer shape of the monitoring device 2. In that embodiment, the ventilated area is about 73 percent of the projected area of the monitoring device 2.

The above mentioned absolute values of the respective projected areas resemble only exemplary values and are only meant for the better understanding of the relation between projected areas of the apertures 14 and the projected area of the monitoring device.

The subject of the invention is not limited to the embodiments described above. Rather, further embodiments of the invention can be derived by the skilled person from the above description. In particular, the individual features of the invention described on the basis of the various embodiment examples and the embodiment variants thereof can also be combined with one another in other ways.

### LIST OF REFERENCE NUMERALS

1 retaining device
2 monitoring device
4 holding frame
6 adhesive layer
8 baseplate
10 snap-on hook
12 overhanging structure
14 aperture
16 through-hole
18 ventilation channel
20 upper side
22 rim
24 plaster
26 adhesive film
30 monitoring system
32 indentation
34 holding magnet
36 floppy ear
38 web
40 stem
42 gap

1. A retaining device for retaining a wearable monitoring device for gathering a bio-electrical signal from a body region of a person, comprising a holding frame having a baseplate and retaining means for reversibly retaining the monitoring device onto the baseplate,
wherein an area covered by the baseplate is reduced with respect to an area covered by the monitoring device for ventilation of the skin during wearing the monitoring device.

2. The retaining device according to claim 1,
the holding frame has a number of apertures penetrating the baseplate.

3. The retaining device according to claim 1,
wherein the baseplate has a skeleton-like or meshed structure of interconnected bars outlining the number of apertures.

4. The retaining device according to claim 2,
wherein the baseplate comprises means for ventilation of at least some of the number of apertures that are located at an upper side of the baseplate facing the monitoring device during intended use.

5. The retaining device according to claim 4,
wherein the means for ventilation comprise at least one ventilation channel embossed into the upper side of the baseplate and connecting one of the number of apertures with a rim of the baseplate.

6. The retaining device according to claim 4,
wherein the means for ventilation comprise at least one protrusion serving as a spacer between the surface of the upper side and the surface of the monitoring device being directed to the baseplate during intended use.

7. The retaining device according to claim 1,
wherein the baseplate has a beam-like structure connecting the retaining means leaving a number of recesses open to a border defined by the area covered by the monitoring device.

8. The retaining device according to claim 1,
wherein a ventilated area which is not covered by the baseplate has at least a portion of 25 percent with respect to the area covered by the monitoring device and a maximum portion of 85 percent, especially a portion between 40 and 75 percent.

9. The retaining device according to claim 1,
wherein the retaining means comprise a snap-on hook.

10. The retaining device according to claim 1,
wherein the retaining means comprise a holding magnet.

11. The retaining device according to claim 1,
wherein the retaining means comprise an overhanging structure into the clearing of which the monitoring device is inserted during intended use.

12. The retaining device according to claim 11,
wherein the overhanging structure is positioned in a region of the baseplate facing upwards during intended use.

13. The retaining device according to claim 9,
wherein the snap-on hook is provided at a rim confining a heel part of the baseplate being tapered with respect to a remaining main part of the baseplate.

14. The retaining device according to claim 1,
comprising means for preventing an unintended allocation of the monitoring device to the holding frame.

15. The retaining device according to claim 14,
wherein the means for preventing an unintended allocation of the monitoring device comprise the baseplate having a curvature, especially the curvature being adapted to a curvature of the monitoring device.

16. The retaining device according to claim 14,
wherein the means for preventing an unintended allocation of the monitoring device comprise the snap-on hook having a length shorter than the thickness of the monitoring device and different to half of the thickness of the monitoring device.

17. The retaining device according to claim 1,
an adhesive layer attached at least to a part of the baseplate for fixation of the holding frame onto the skin of the person, especially wherein the adhesive layer has a multiple layer structure comprising a carrier layer attached by an adhesive sublayer to the baseplate and a contact layer that serves as a skin adhesive during intended use.

18. The retaining device according to claim 17,
wherein the adhesive layer has a number of through-holes corresponding to the respective aperture of the baseplate.

19. A wearable monitoring system comprising a wearable monitoring device for gathering a bio-electrical signal and a retaining device according to claim 1.
